SEQUENCE LISTING (17 sequences)

SEQ ID NO: 1 - protein, 117 aa, synthetic construct
EVQLQESGPELVKPGASVKLSCKASGYTFTSYDINWVKQRPGQGLEWIGWIFPRDGYTKYNEKFKGKATLTVDTSSSTAYMELHSLTSEDSAVYFCARYYNWYFDVWGTGTTVTVSS

SEQ ID NO: 2 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 3 - protein, 120 aa, synthetic construct
EVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 4 - protein, 106 aa, synthetic construct
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELK

SEQ ID NO: 5 - protein, 124 aa, synthetic construct
QVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSS

SEQ ID NO: 6 - protein, 112 aa, synthetic construct
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 7 - protein, 120 aa, synthetic construct
EVQLQQSGPVLVKPGASVKMSCKASGYTFTDYYMNWVKQSHGKSLEWIGVINPYNGATAFNQKFKGKATLTVDKSSSTAYMDLTSLTSEDSAVYYCARDDGYYLYYFDYWGQGTTLTVSL

SEQ ID NO: 8 - protein, 107 aa, synthetic construct
DIQMTQSPSSLSASLGERVSLTCRASQDIGSRLNWLQQEPDGTIKRLIYATSSLDSGVPKRFSGSRSGSDYSLTISSLESEDFVDYYCLQYASSPPTFGGGTKLEIK

SEQ ID NO: 9 - protein, 119 aa, synthetic construct
QIQLVQSGPELKKPGETVKISCKASGYTFTTYGMSWVKQAPGKGLKWMGWINTYSGVPTYADDFKGRFAFSLETSASTAYLQINNLKNEDTATYFCARDPYYSSGWNYWGQGTTLTVSS

SEQ ID NO: 10 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 11 - protein, 117 aa, synthetic construct
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAVRPFYAMDYWGQGTSVTVSS

SEQ ID NO: 12 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 13 - protein, 122 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARIVGTTVVAPFAYWGQGTLVTVSA

SEQ ID NO: 14 - protein, 111 aa, synthetic construct
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 15 - protein, 117 aa, synthetic construct
EVNLEESGGGLVQPGGSMKLSCVASGFTFSNYWMNWVRQSPEKGLEWLAQIRLKSDNYVIYYAESVKGRFTISRDDSKSSVYLQMNNLRAEDTGIYYCIRGYFFDYWGQGTTLTVSS

SEQ ID NO: 16 - protein, 107 aa, synthetic construct
DIQMTQTTSSLSASLGDRVTISCSASQGISNYLNWYQQKPGGTVKLLIYYTSNLHSGVPSRFSGSGSGTDYSLTISNLEPEDIATYYCQQYSKLPYTFGGGTKLEIK

SEQ ID NO: 17 - protein, 8 aa, synthetic construct
SIINFEKL

(19) United States
(12) Patent Application Publication
(10) Pub. No.: US 2026/0167722 A1
(43) Pub. Date: Jun. 18, 2026

(54) LYMPHOCYTE-ACTIVATING GENE 3 (LAG-3) TARGETED T CELL SILENCER FOR THE TREATMENT OF AUTOIMMUNE DISEASES

(71) Applicant: New York University, New York, NY (US)

(72) Inventors: Jun WANG, New York, NY (US); Jasper DU, New York, NY (US); Jia YOU, New York, NY (US)

(21) Appl. No.: 19/124,631

(22) PCT Filed: Oct. 27, 2023
(86) PCT No.: PCT/US2023/078092

§ 371 Date: Apr. 25, 2025

Related U.S. Application Data
(60) Provisional application No. 63/381,257, filed on Oct. 27, 2022.

(51) Int. Cl. C07K 16/28; A61K 39/00; A61P 37/06

(52) U.S. Cl. CPC C07K 16/2809; A61P 37/06; C07K 16/2803; A61K 2039/505; C07K 2317/31; C07K 2317/622; C07K 2317/75

(57) ABSTRACT

Provided are compositions and methods for prophylaxis or therapy of autoimmune disorders. The compositions comprising binding partners configured to locate LAG-3 and a T cell receptor closer to one another than in the absence of the binding partner to thereby provide for an immunosuppressive effect on activated T cells.

### CROSS-REFERENCE TO RELATED APPLICATION

This application claims priority to U.S. provisional application No. 63/381,257, filed Oct. 27, 2022, the entire disclosure of which is incorporated herein by reference.

### FIELD

The present disclosure relates generally to compositions and methods for prophylaxis or therapy for autoimmune diseases, and more specifically to a new approach to suppressing the function of T cells that contribute to autoimmune diseases.

### SEQUENCE LISTING

The instant application contains a Sequence Listing, which is submitted in .xml format and is hereby incorporated by reference in its entirety. Said .xml file is named “058636_00646_ST26.xml”, was created on Oct. 27, 2023, and is 19,476 bytes in size.

### RELATED INFORMATION

T cells are essential components of the human adaptive immune system, which can directly mediate tissue damage when recognize autoantigens, and also modulate the responses of other immune cells, such as B cell's autoreactive antibody generation through T helper cells, or T regulatory cells. T cells have an important function in the disease pathogenesis and progression of many autoimmune diseases, such as multiple sclerosis, arthritis, diabetes, and Systemic Lupus Erythematosus (SLE), and others as described herein. However, taming T cells for autoimmune diseases therapy requires a delicate and well-coordinated control, as the dampening of T cell immunity could simultaneously lead to severe immunodeficiency diseases. For instance, general immune suppression by steroids provides only symptomatic relief and do not eliminate the cause of the pathology and are often associated with significant side-effects and infections in various autoimmune diseases. Thus, there is an ongoing an unmet need for compositions and methods that specifically target disease-causing autoreactive T cells while sparing other T cell subsets that are beneficial for immune defense and homeostasis. The present disclosure is pertinent to this need.

### BRIEF SUMMARY

The present disclosure provides compositions and methods for use in treatment or prophylaxis of autoimmune disorders. The compositions include a binding partner having a first binding component that binds with specificity to a T cell receptor (TCR) component or other protein that is near a TCR component, and a second component that binds with specificity to LAG-3. Binding of the binding partner suppresses activity of the T cell insofar as the T cell participates in promoting one or more symptoms of an autoimmune disorder. In some embodiments the compositions are provided as a bispecific antibody-based T cell silencer (BiTS). Polynucleotides that encode the bispecific binding partners are included. The polynucleotides may be DNA, including but not limited to a cDNA, and may be present in any type of expression vector, or may be RNA. The disclosure includes administering to an individual who has an autoimmune disorder a described binding partner, or a polynucleotide encoding a described binding partner. It is considered that administering a polynucleotide that results in expression of the binding partner is also an administration of the binding partner itself. Administration of a described binding partner can be used prophylactically and therapeutically. In embodiments, administration of a described binding partner results in inhibition of the progression of the autoimmune disorder, or inhibition of development of the autoimmune disorder, or inhibition of autoimmune disorder relapse. Cells that are engineered to express a described binding partner are included in the disclosure. Such cells include but are not necessarily limited to T cells, natural killer cells, macrophages, T cell receptor engineered cells, and the like. In embodiments, any of the modified cells express a described binding partner in a chimeric antigen receptor format.

### BRIEF DESCRIPTION OF FIGURES

FIG. 1. Schematic illustration of LAG-3-mediated inhibition of T cell responses.

FIG. 2. Panel A) Left: Schematic of the classical in vitro assay to evaluate LAG-3 function on mouse T cells. In this assay, MHC-II from mouse LK35.2 B cells simultaneously binds both mouse T cell receptor (TCR) and mouse LAG-3 in the presence of HEL peptide, and 3A9 T cell hybridoma cells express cognate TCR for MHC-II/HEL complex. Right: Restoration of T cell activation using a known LAG-3 antagonist, C9B7W. Panel B) Left: Schematic of an artificial antigen presenting cell (APC) approach capable of isolating the MHC-II/LAG-3 interaction. Membrane tethered anti-mouse CD3 (145.2C11) single chain variable fragment expressed on 293T cells is used to activate the T cells, and MHC-II bound to a non-cognate OVA peptide only binds to LAG-3 but not the TCR of 3A9 T cells. Right: Evaluation of LAG-3 antagonist, C9B7W, in this artificial antigen presenting cell (APC) approach.

FIG. 3. Panel A) Schematic of a mouse artificial antigen presenting cell (APC) approach capable of activating mouse 3A9 T cells containing a NFAT-GFP reporter. Panel B) Generation of artificial APCs containing different expression levels of the membrane tethered single chain variable fragment (scFv) of the anti-mouse CD3 antibody 145.2011. Median fluorescence intensities of three different membrane tethered αCD3 293T clones. Panel C) IL-2 secretion after co-culture of different αCD3 clones with 3A9 T cells. Panel D) Evaluation of different αCD3 clones when cocultured with T cells containing the NFAT-GFP reporter. Fluorescence was quantified from reporter cell activation using images taken by CellInsight CX7.

FIG. 4. Panel A) Evaluation the function of MHC-II fused with non-cognate peptide expressed on different αCD3 APC clones on LAG-3⁺ 3A9 T cells as measured by IL-2 after 24 hours. Panel B) Co-culture of αCD3 low APC in the presence or absence of MHC-II (non-cognate) to evaluate the individual contribution of MHC-II to LAG-3 signaling (with or without anti-LAG-3 C9B7W).

FIG. 5. Panel A) Schematic of a proposed rapalog system where the rapalog can induce heterodimers of aCD3e and MHC-II_{(non-cognate)} thereby forcing proximity between the TCR-CD3 complex and LAG-3. Panel B) Evaluation of the rapalog heterodimer system to induce proximity between the TCR-CD3 complex and LAG-3, triggering LAG-3 mediated T cell inhibition. T cell activation was measured by IL-2 after 24 hours of co-culture.

FIG. 6. Panel A) Schematic of the LAG-3-CAR-T system to evaluate engagement of LAG-3 signaling by either membrane or soluble LAG-3 ligands. Panel B) Quantification of fluorescence from LAG-3 CAR-T NFκB GFP Jurkat reporter cell activation by the putative ligands of LAG-3 using images obtained using CellInsight CX7. Plasmids representing each ligand were transfected into HEK-293T cells and subsequently co-cultured with LAG-3 CAR-T NFκB-GFP Jurkat reporter cells. Panel C) Activation of LAG-3 CAR-T by two different forms of soluble FGL1, oligomer and dimer.

FIG. 7. Panel A) LAG-3 extracellular domain mapping of the anti-mouse LAG-3 antibody M8. Panel B) Binding epitope of the anti-mouse LAG-3 antibody M8. Key residues for M8 binding to domain 1 of LAG-3 are indicated (PDB: 7TZE).

FIG. 8. Panel A) Evaluation of MHC-II and FGL1 blocking capability of M8 (10 ug/ml) in the LAG-3 CAR-T assay. Panel B) Evaluation of anti-LAG-3 antibodies in a LK35.2/3A9 co-culture assay. LK35.2 cells were pulsed with 1 μM HEL peptide and co-cultured with T cells in the presence of either isotype or anti-mouse LAG-3 M8 or C9B7W antibody (10 ug/mL). T cell activation was measured by IL-2 after 24 hours of co-culture. Panel C) Evaluation of the anti-LAG-3 antibodies C9B7W and M8 in an MC38 tumor model. Mice were treated with indicated antibody at 100 ug per mouse since day 6 for 4 doses (twice a week).

FIG. 9. Antibody variable domain sequences of M8 with the CDR regions bolded. The sequence of the M8 heavy chain is SEQ ID NO:1. The sequence of the M8 light chain is SEQ ID NO:2. Non-limiting embodiments of the disclosure are demonstrated using H57 antibody sequences in a bi-specific format that also contains M8 antibody sequences.

FIG. 10. Panel A) Schematic of TCR/LAG-3 (mouse/mouse) bispecific antibody (Bispecific T cell silencer, BiTS) used to induce proximity between MHC-II and LAG-3. A mutated version with retained binding to TCR but no binding to LAG-3 is shown as a control. Panel B) Binding of H57xM8 BiTS and mutant to TCR and LAG-3.

FIG. 11. Evaluation of H57xM8 BiTS and mutant in the inhibition of T cell responses. Panel A) CD4⁺ (3A9, bearing TCR reactive to MHC-II-HEL peptide) or CD8⁺ (B3Z, bearing TCR reactive to MHC-I-OVA peptide) T cell hybridoma cells with or without LAG-3 overexpression were stimulated with membrane tethered anti-CD3 and treated with isotype (human IgG1), H57xM8 BiTS, or H57xM8 BiTS₍ₘᵤₜ₎. T cell activation was measured by IL-2 after 24 hours of co-culture. The % inhibition of T cell activation was calculated using the isotype control as a reference. Panel B) 3A9 or B3Z T cells were stimulated with cognate peptide MHC (MHC-II-HEL for 3A9 or MHC-I-OVA for B3Z), respectively, and treated with isotype (human IgG1), H57xM8 BiTS, or H57xM8 BiTS₍ₘᵤₜ₎. T cell activation was measured by IL-2 after 24 hours of co-culture. The % inhibition of T cell activation was calculated using the isotype control as a reference. Panel C) Evaluation of H57xM8 BiTS and mutant in an ex vivo CD8⁺ OT-I primary T cell system. Naïve OT-1 T cells bearing OVA TCR with WT or Lag3^{-/-} background were isolated and stimulated with an mutuDC cell line pulsed with SIINFEKL peptide (1 ng/ml). T cell activation was measured by IL-2 after 24 hours and IFNγ after 120 hours of co-culture. Panel D) Dose response curve of H57xM8 BiTS in this ex vivo OT-I T cell system. T cell activation was measured by IL-2 after 48 hours of co-culture.

FIG. 12. Panel A) Schematic of TCR/LAG-3 (mouse/human) bispecific antibody used to induce proximity between TCR and human LAG-3. Evaluation of H57xRE (relatlimab) BiTS in either a CD4⁺ (3A9) or CD8⁺ (B3Z) T cell system (Panel B). Human LAG-3⁺ 3A9 or B3Z T cells were stimulated with either anti-CD3 or MHC-I-OVA, respectively, and treated with BiTS (10 ug/mL). T cell activation was measured by IL-2 after 24 hours of co-culture.

FIG. 13. Evaluation of H57xM8 BiTS in an experimental autoimmune encephalomyelitis (EAE) model. Two treatment regiments were employed: Panel A) prophylactic, and Panel B) therapeutic. C57BL/6 mice were immunized with myelin oligodendrocyte glycoprotein (MOG), injected i.p. with pertussis toxin on day 0 and day 2, then treated with either PBS or BiTS i.p. at 1.25 mg/kg. For prophylactic treatment, mice were treated daily from day 6 to day 10 before the onset of symptoms. For therapeutic treatment, mice were treated every other day from day 13 to 21.

FIG. 14. Evaluation of H57xM8 BiTS in a coreceptor absent system. Mouse LAG-3⁺ CD4 KO 3A9 and CD8 KO B3Z cells were generated using CRISPR-Cas9. Panel A) LAG-3⁺ 3A9 CD4 KO cells were stimulated with membrane tethered anti-CD3 and treated with BiTS (1 ug/ml). Panel B) LAG-3⁺ B3Z CD8 KO cells were stimulated with mutuDC cells with indicated SIINFEKL peptide and treated with BiTS (1 ug/ml).

FIG. 15. Evaluation of OKT3xRE (relatlimab) BiTS in a Jurkat NFκB GFP reporter system. Panel A) Schematic of the Jurkat NFκB GFP reporter system. Panel B) Design of the OKT3xRE BiTS. Panel C) LAG-3⁺ Jurkat cells bearing a gp100-specific TCR were activated with MHC-I-gp100 expressed on 293T and treated with BiTS (5 ug/ml).

FIG. 16. Evaluation of OnoxRE (relatlimab) BiTS in a Jurkat NFAT GFP reporter system. Panel A) Schematic of the Jurkat NFAT GFP reporter system. Panel B) Design of the OnoxRE BiTS. Panel C) LAG-3⁺ Jurkat cells bearing a HA₍₃₀₆₋₃₁₈₎ specific TCR were activated with DR1 HA₍₃₀₆₋₃₁₈₎ expressed on 293T and treated with BiTS (5 ug/ml).

FIG. 17. Panel A) Binding epitope of the anti-mouse TCR antibody H57-597 (PDB: 1NFD). Key residues for H57-597 binding to the TCRβ constant chain are indicated. Panel B) H57-597 antibody variable domain sequences with the CDR regions bolded. SEQ ID NOs: 18 and 19 are shown.

FIG. 18. Evaluation of H57xNivo (Nivolumab) BiTS in a human PD-1⁺ LAG-3⁻ CD4⁺ T cell (3A9) or CD8+ T cell (B3Z) system. Panel A) Design of the H57xNivo BiTS. Panel B) 3A9 and B3Z cells were stimulated with either membrane tethered anti-CD3 or mutuDC cells with SIINFEKL (SEQ ID NO:17) peptide (2 ng/ml), respectively, and treated with BiTS (1 ug/ml).

FIG. 19. Evaluation of H57xM8 BiTS in a RIP-OVA autoimmune diabetes model. Panel A) Experimental scheme for the RIP-OVA model. OT-I T cells (300,000) activated ex vivo were injected i.v. into RIP-OVA mice at day 0 and treated with either PBS, H57xM8 BiTS, or the mutant H57xM8 BiTS every other day from day 0 to day 8 at 1.25 mg/kg. Diabetes was defined as >250 mg/dL blood glucose for 3 consecutive days. Panel B) Diabetes incidence in RIP-OVA mice treated with either PBS, H57xM8 BiTS₍ₘᵤₜ₎, or H57xM8 BiTS (n=8). Panel C) Representative histological images of pancreatic islets indicating no insulitis, peri-insulitis, and insulitis. Panel D) Histological assessment of insulitis in surviving RIP-OVA mice at day 14. At least 10 pancreatic islets were graded per mouse.

FIG. 20. Evaluation of H57xM8 BiTS in an anti-41BB autoimmune hepatitis model. Panel A) Experimental scheme for the hepatitis model. C57BL/6 mice were injected with 100 ug anti-41BB i.p. on day 0 and day 7 and treated with either PBS or H57xM8 BiTS daily from day 6 to day 10 at 1.25 mg/kg. Liver sections, liver homogenates, and serum were collected for histological, cytokine, and ALT analysis, respectively. Panel B) Representative histological images from liver sections from healthy control, PBS, and H57xM8 BiTS treatment groups. Panels C-F) ALT, IFNγ, TNFα, and MCP-1 measurements from healthy control, PBS, and H57xM8 BiTS treatment groups (n=4).

FIG. 21. Evaluation of H57xM8 BiTS on different CD4/CD8 subsets within the liver in an anti-41BB autoimmune hepatitis model. Intrahepatic lymphocytes were stained for several T cell surface markers to determine the depleting effects of H57xM8 BiTS in comparison to healthy control, PBS treated, and mutant H57xM8 BiTS treated groups. Panel A) CD45, CD8, and CD4. Panel B) CD45, CD4, CD8, and LAG-3. Panel C) CD45, CD8, and PD-1.

FIG. 22. Evaluation of H57xM8 BiTS in an anti-41BB autoimmune hepatitis model. C57BL/6 mice were injected with 100 ug anti-41BB i.p. on day 0 and day 7 and treated with either PBS, H57xM8 BiTS, or the mutant H57xM8 BiTS daily from day 6 to day 10 at 1.25 mg/kg. Panels A-D) Liver homogenates were collected for IFNγ, TNFα, MCP-1, and IL-12p70 cytokine analysis (n=5).

FIG. 23. Overview Figure. This figure provides a non-limiting schematic illustration of an approach provided by the present disclosure.

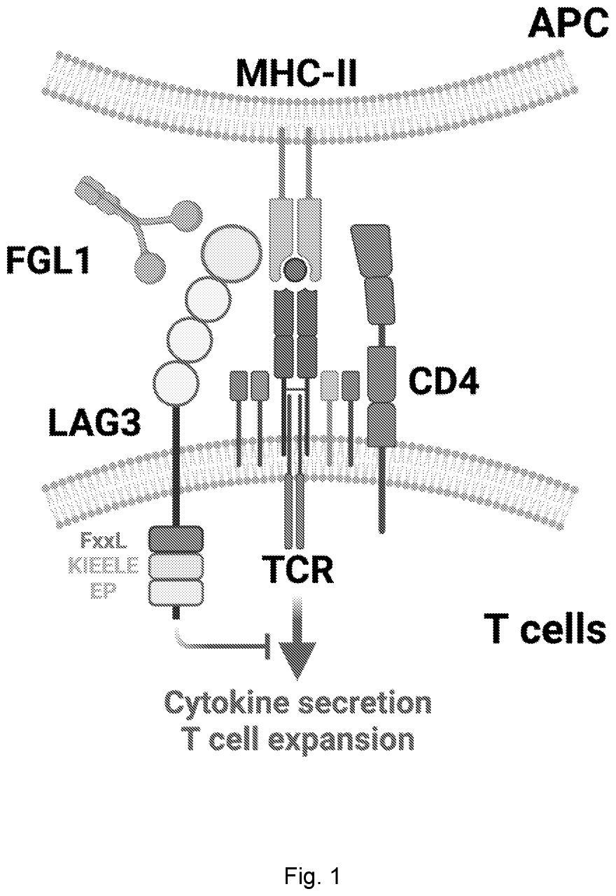

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

This disclosure includes every amino acid sequence described herein and all nucleotide sequences encoding the amino acid sequences. Every antibody sequence and antigen binding fragments of them are included. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences.

The present disclosure, among other aspects, reveals previously unknown features that affect T cells when LAG-3 is positioned proximal to a component of a TCR, or proximal to one or more proteins that are normally positioned on a T cell surface near a TCR. The disclosure demonstrates a dampening effect on TCRs that are involved in deleterious immune responses using bispecific binding partners that concurrently specifically bind to LAG-3 and specifically bind to a TCRβ chain. Based on this demonstration it is expected that binding partners that bind to other components of a TCR complex or other proteins near a TCR will have a similar effect on T cells. Thus the disclosure includes using binding partner that comprises a first binding component that binds with specificity to LAG-3, and a second binding component that binds to a component of a TCR complex, such as a TCRβ chain, or TCR gamma or delta chains, or proteins that are normally located near a TCR complex, including but not necessarily limited to CD3δ, CD3γ, CD3ε and CD3ζ, CD4, CD5, CD6, CD7, and CD8.

Non-limiting embodiments of the disclosure are demonstrated using the H57 antibody as a component of a described binding partner. The terms “H57” and “H57-597” as used in the specification refer to the same antibody construct, which specifically binds to mouse TCR beta chain. The H57 antibody is known in the art and is commercially available, such as from BioXCell Catalog #BE0102. Additional TCR binding antibodies are known in the art and include the BMA-031 antibody

having the BMA-031 Heavy chain sequence
(SEQ ID NO: 3)
EVQLQQSGPELVKPGASVKMSCKASGYKFTSYVMHWVKQKPGQGLEWIGY
INPYNDVTKYNEKFKGKATLTSDKSSSTAYMELSSLTSEDSAVHYCARGS
YYDYDGFVYWGQGTLVTVSA
and
the BMA-031 Light (κ) sequence
(SEQ ID NO: 4)
QIVLTQSPAIMSASPGEKVTMTCSATSSVSYMHWYQQKSGTSPKRWIYDT
SKLASGVPARFSGSGSGTSYSLTISSMEAEDAATYYCQQWSSNPLTFGAG
TKLELK
and
the JOVI-1 antibody having the heavy chain
sequence
(SEQ ID NO: 5)
QVQLVQSGAEVKKPGASVKVSCKASGYTFTGYVMHWVRQAPGQGLEWMGF
INPYNDDIQSNERFRGRVTMTRDTSISTAYMELSRLRSDDTAVYYCARGA
GYNFDGAYRFFDFWGQGTMVTVSS
and
the JOVI-1 Light (κ) chain sequence
(SEQ ID NO: 6)
DIVMTQSPLSLPVTPGEPASISCRSSQRLVHSNGNTYLHWYLQKPGQSPR
LLIYRVSNRFPGVPDRFSGSGSGTDFTLKISRVEAEDVGVYYCSQSTHVP
YTFGQGTKLEIK.

In embodiments, the component of a described construct that specifically binds to LAG-3 comprises a LAG-3 specific antibody, examples of which are known in the art, and include but are not necessarily limited to antibodies known as IMP761, MGD013, BI754111, XmAb 22841, Sym022, MK-4280, TSR-033, REGN3767, GSK2831781, LBL-007, LAG525, INCAGN02385, and Relatlimab sold under the tradename OPDUALAG. In embodiments, use of a described bispecific construct as described herein exhibits improved immunosuppressive effects compared to using a construct that binds to LAG-3 alone, such as is described in Angin et al. J Immunol (2020) 204 (4): 810-81, or a LAG-3 binding agent that does not bring the LAG-3 proximal to a TCR chain. In a non-limiting embodiment, use of described a bispecific construct induces more inhibition of T cell activation than a construct that binds to LAG-3 alone, or a LAG-3 binding agent that does not bring the LAG-3 proximal to a TCR chain.

In addition to the known antibodies, the disclosure includes the following antibody heavy and light chain sequences for use as an anti-human LAG-3 component for use in prophylaxis and/or therapy of autoimmune conditions:

H4-10 Heavy Chain Variable Domain
(SEQ ID NO: 7)
EVQLQQSGPVLVKPGASVKMSCKASGYTFTDYYMNWVKQSHGKSLEWIGV
INPYNGATAFNQKFKGKATLTVDKSSSTAYMDLTSLTSEDSAVYYCARDD
GYYLYYFDYWGQGTTLTVSL
H4-10 Light Chain Variable Domain (κ)
(SEQ ID NO: 8)
DIQMTQSPSSLSASLGERVSLTCRASQDIGSRLNWLQQEPDGTIKRLIYA
TSSLDSGVPKRFSGSRSGSDYSLTISSLESEDFVDYYCLQYASSPPTFGG
GTKLEIK
H3-6 Heavy Chain Variable Domain
(SEQ ID NO: 9)
QIQLVQSGPELKKPGETVKISCKASGYTFTTYGMSWVKQAPGKGLKWMGW
INTYSGVPTYADDFKGRFAFSLETSASTAYLQINNLKNEDTATYFCARDP
YYSSGWNYWGQGTTLTVSS
H3-6 Light Chain Variable Domain (κ)
(SEQ ID NO: 10)
DIVMTQSQKFMSTSVGDRVSVTCKASQNVGTNVAWYQQKPGQSPKALIYS
ASYRYSGVPDRFTGSGSGTDFTLTISNVQSEDLAEYFCQQYNSYPYTFGG
GTKLEIK
H8 Heavy Chain Variable Domain
(SEQ ID NO: 11)
EVQLQQSGPELVKPGASVKISCKASGYTFTDYYMNWVKQSHGKSLEWIGD
INPNNGGTSYNQKFKGKATLTVDKSSSTAYMELRSLTSEDSAVYYCAVRP
FYAMDYWGQGTSVTVSS
H8 Light Chain Variable Domain (κ)
(SEQ ID NO: 12)
DIVLTQSPASLAVSLGQRATISCRASESVDNYGISFMNWFQQKPGQPPKL
LIYAASNQGSGVPARFSGSGSGTDFSLNIHPMEEDDTAMYFCQQSKEVPW
TFGGGTKLEIK
H12 Heavy Chain Variable Domain
(SEQ ID NO: 13)
QVTLKESGPGILQPSQTLSLTCSFSGFSLSTFGMGVGWIRQPSGKGLEWL
AHIWWDDDKYYNPALKSRLTISKDTSKNQVFLKIANVDTADTATYYCARI
VGTTVVAPFAYWGQGTLVTVSA
H12 Light Chain Variable Domain (κ)
(SEQ ID NO: 14)
DIVLTQSPASLAVSLGQRATISCRASESVDSYGNSFMHWYQQKPGQPPKL
LIYRASNLESGIPARFSGSGSRTDFTLTINPVEADDVATYYCQQSNEDPY
TFGGGTKLEIK
M7695 Heavy Chain Variable Domain
(SEQ ID NO: 15)
EVNLEESGGGLVQPGGSMKLSCVASGFTFSNYWMNWVRQSPEKGLEWLAQ
IRLKSDNYVIYYAESVKGRFTISRDDSKSSVYLQMNNLRAEDTGIYYCIR
GYFFDYWGQGTTLTVSS
M7695 Heavy Chain Variable Domain (κ)
(SEQ ID NO: 16)
DIQMTQTTSSLSASLGDRVTISCSASQGISNYLNWYQQKPGGTVKLLIYY
TSNLHSGVPSRFSGSGSGTDYSLTISNLEPEDIATYYCQQYSKLPYTFGG
GTKLEIK

In embodiments, the described binding partner is a bispecific antibody, but other formats are included within the scope of the disclosure, such as tri-specific antibodies. The term “antibody” includes all forms of a binding partner that specifically binds its cognate antigen, including but not limited to segments of antibodies that specifically bind to the cognate antigen. Bispecific antibodies include all forms of bispecific antibodies, including but not necessarily limited to those described in /doi.org/10.3389/fimmu.2021.626616, from which the description is incorporated herein by reference.

With respect to the component of a bispecific antibody that binds to the TCR alpha or beta chains, in general this component will bind to a constant region of one of the chains. However, customized bispecific antibodies that bind to a variable region, including but not necessarily limited to TCR alpha or beta chain complementarity-determining regions (CDRs), are included within the scope of the disclosure, such as for use in a personalized medicine approach.

It is known that LAG-3 is a T-cell checkpoint receptor primarily found on activated T cells, which can be induced by cytokines such as interleukin (IL)-2, IL7, and IL-12. LAG-3 negatively regulates the proliferation, activation, effector function, and homeostasis of both CD8+ and CD4+ T cells. The immuno-suppressive activity of LAG-3 is mediated through its intracellular signaling domain. A secreted protein, Fibrinogen-like Protein 1 (FGL1), is an MHC-II-independent, high affinity LAG-3 ligand. FGL1 is a hepatocyte-secreted protein in the fibrinogen family without a clear link to fibrin clot formation but demonstrates activities in hepatocyte proliferation and liver metabolic function.

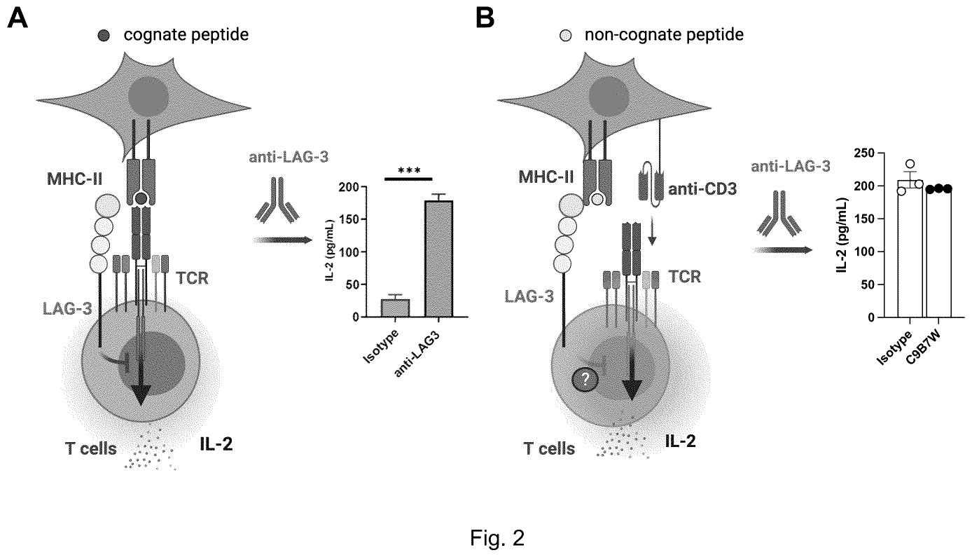

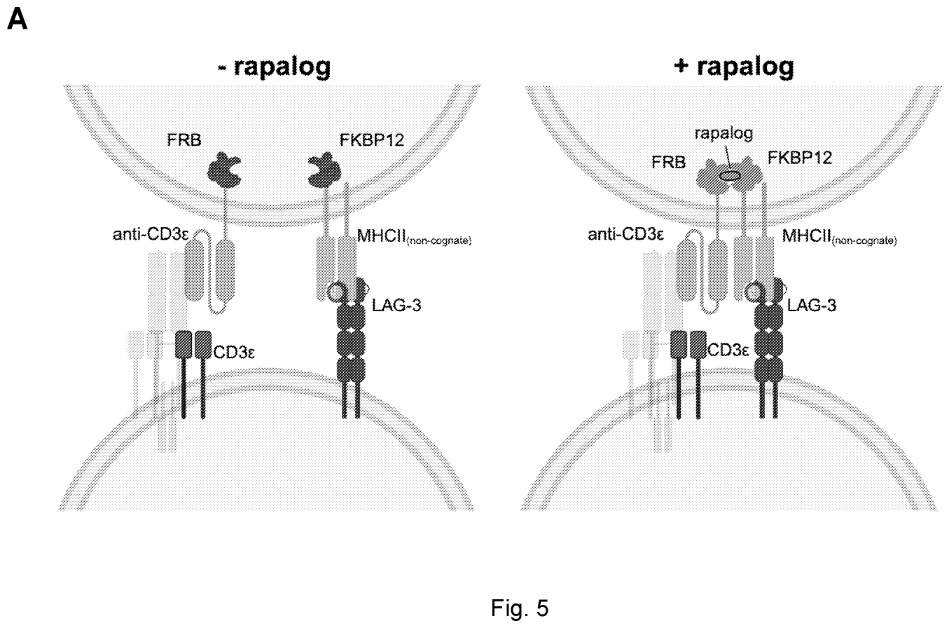

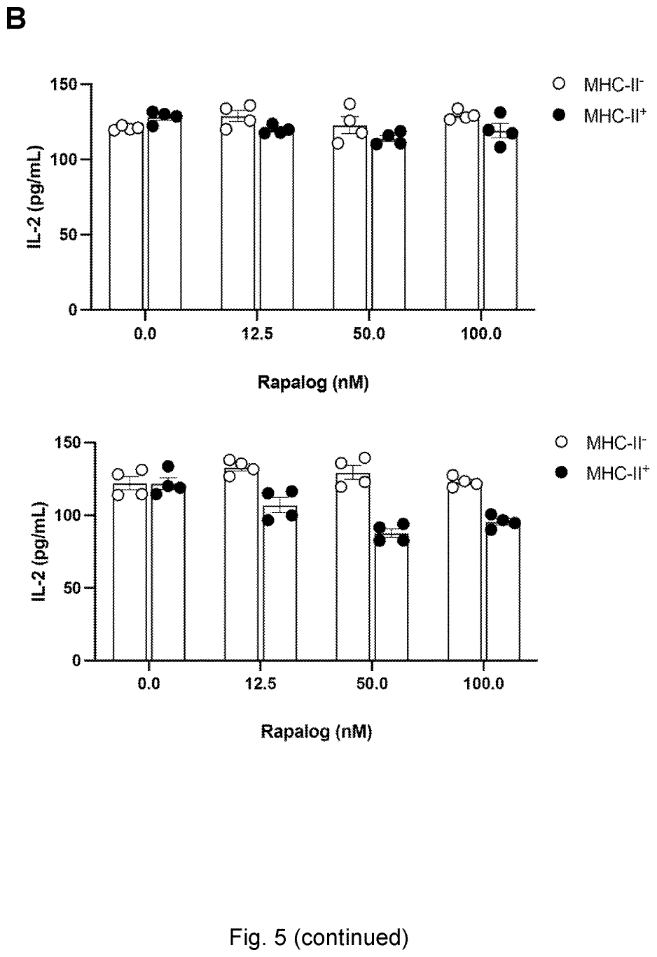

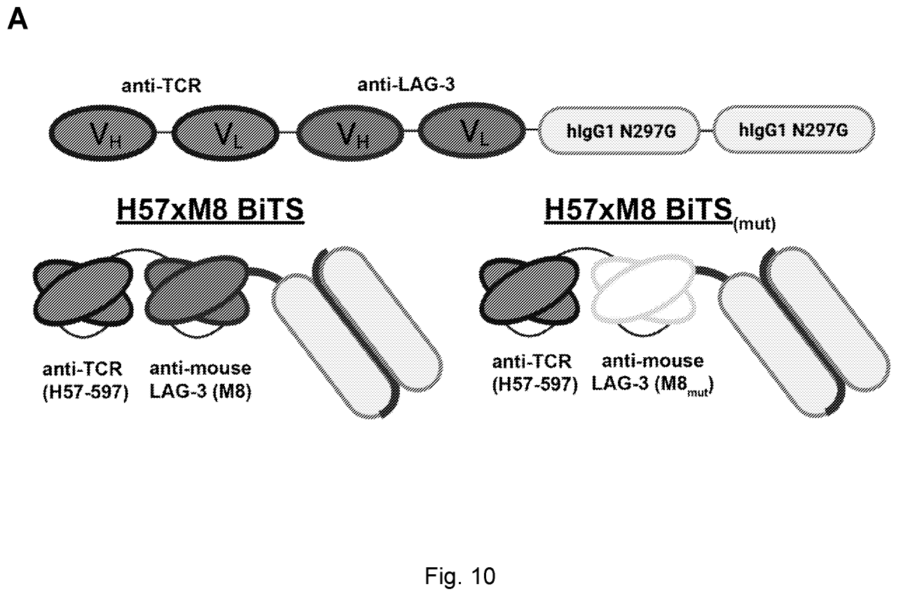

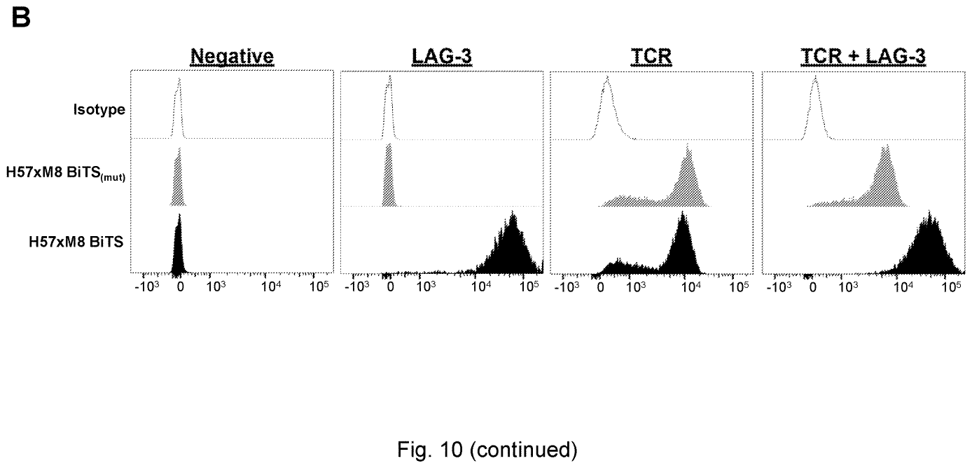

Despite over thirty years of research, the precise mechanism of T-cell inhibition by LAG-3 has remained unclear. To address this knowledge gap, the disclosure provides a designed series of in vitro T cell functional system to specifically define the role of MHC-II/LAG-3 interaction without TCR engagement (FIGS. 2-4). The present disclosure reveals that LAG-3 proximity to the TCR-CD3 complex normally enforced by MHC-II is essential for the function of LAG-3 (FIG. 5). Based in part on this determination, the present disclosure provides representative and non-limiting bispecific antibodies that position LAG-3 more closely to the TCR complex. The antibodies demonstrate strong LAG-3 dependent in vitro activities on both CD4⁺ and CD8⁺ T cells (FIGS. 10-13, 19-22). Given the potent immune suppressive activity of this antibody approach, the antibodies of this disclosure are referred to herein as bispecific T cell silencers (BiTS).

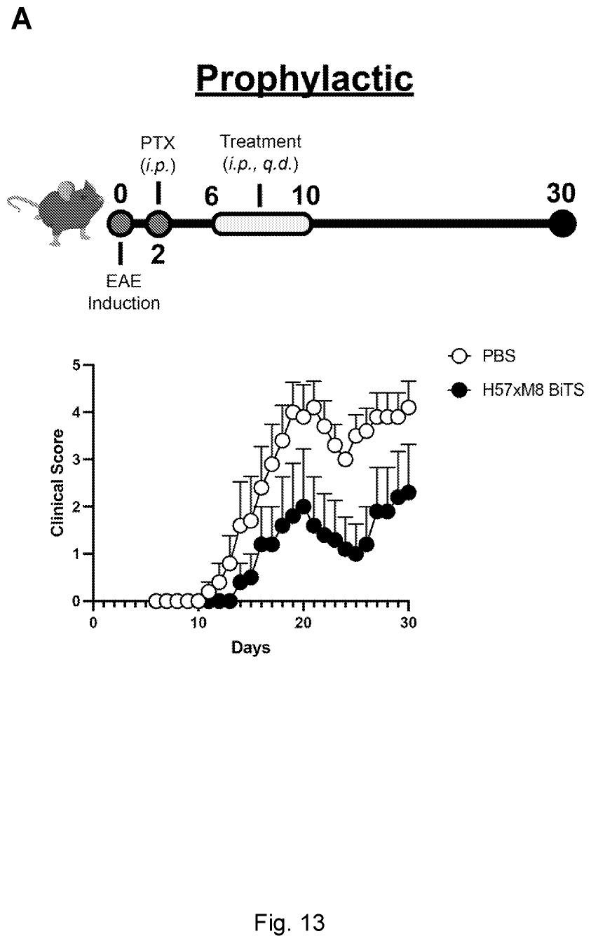

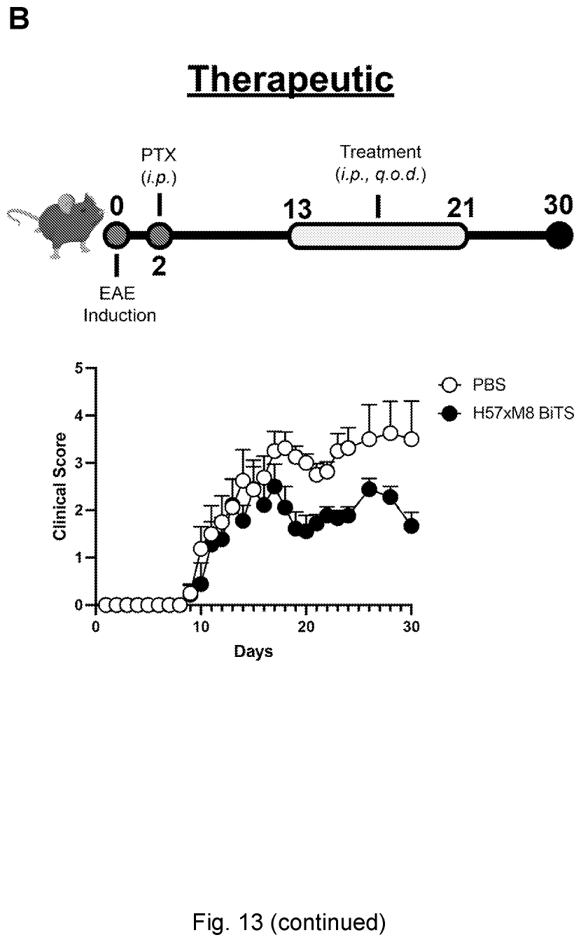

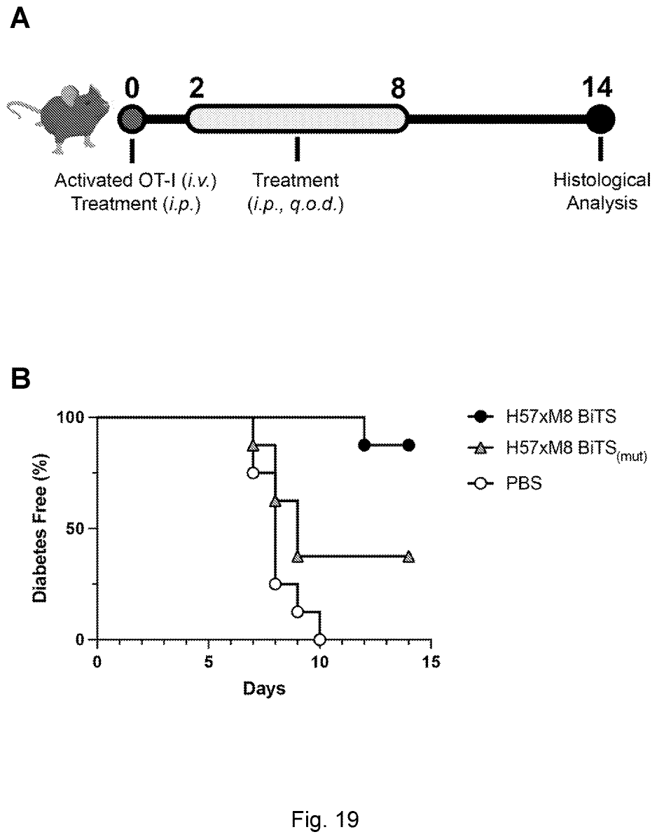

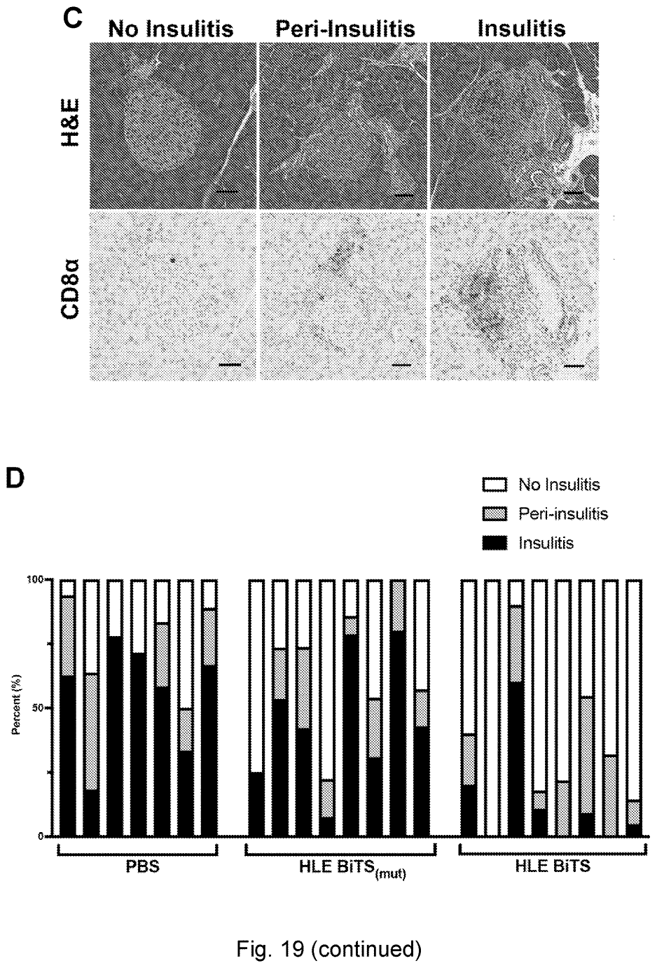

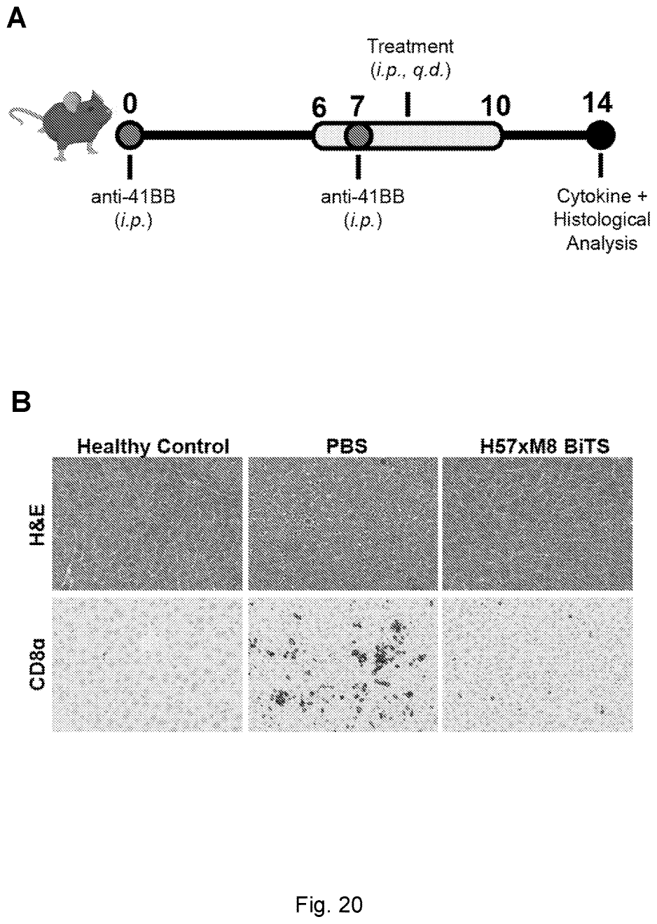

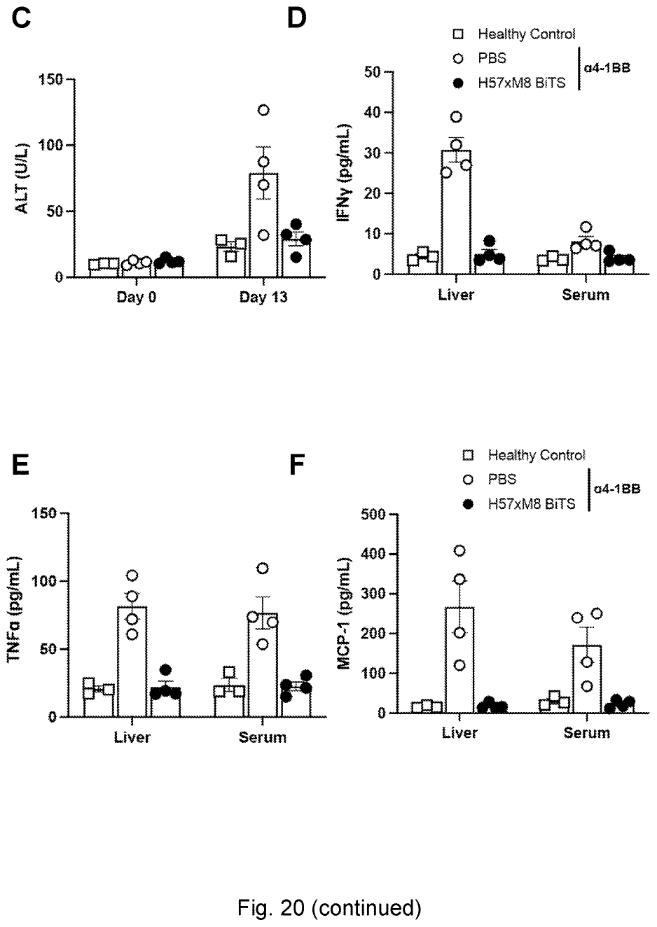

In a non-limiting example, the disclosure demonstrates that the described BiTS significantly ameliorated disease symptoms in experimental autoimmune encephalomyelitis (EAE) model of multiple sclerosis (MS) (FIG. 13), and protects against the development of diabetes (FIG. 19), and protects against hepatitis in an anti-41BB autoimmune hepatitis model (FIG. 20). Other beneficial effects in terms of mitigating autoimmune effects are also demonstrated in the figures. Therefore, the presently provide approach demonstrates use of BiTS as potent T cell checkpoint agonists for treating MS and diabetes, which is expected to be extendable to other autoimmune diseases, as described further below.

It will be recognized from the present description and figures that the disclosure provides unique in vitro T cell functional assay systems that were used in determination of the specific role of MHC-II/LAG-3 interaction without simultaneous TCR triggering. The finding of the importance of LAG-3 proximity to TCR, and small molecule-driven proximity to re-enforce LAG-3 function are believed to be novel. In this regard, a recent publication suggest that LAG-3 will mediate T cell suppression by tonic signaling, preventing Lck from interacting with the co-receptors CD8 and CD4 in the immune synapse (PMID: 35437325), possibly in an MHC-II independent manner, although FGL1 may still play a role in this system. In contrast, the presently provided data indicate that TCR proximity, but not the co-receptor engagement, is an important factor for LAG-3 mediated immune suppression, and this mechanism can be used to design LAG-3 based checkpoint agonists, representative examples of which are described and used in the Figures and data of this disclosure. Without intending to be bound by any particular theory, it is also considered that ligand engagement by MHC-II is required due to the ability of MHC-II to simultaneously bind both TCR and LAG-3. The present disclosure therefor supports the interpretation that MHC-II, and potentially the oligomeric form of FGL1, enhances proximity between the TCR-CD3 complex and LAG-3, and LAG-3 acts upon this complex to carry out its inhibitory function. The present disclosure therefore challenges the traditional dogma in the co-signaling receptor field that immune receptor signal is triggered only upon trans-interaction with membrane ligand. Instead, the disclosure reveals that another level of TCR proximity control in cis is required for the receptor signal. Previously available autoimmune disease treatment has primarily focused on cytokines or B cell modulatory agents. The present disclosure accordingly provides an alternative to these approaches by demonstrating T cell-based immunotherapy by targeting activated T cells while sparing naïve T cells. The described BiTS is therefore considered to represent a unique checkpoint agonist for the treatment of autoimmune disease, given its selective but potent activity in suppressing both CD4+ and CD8+ LAG-3 positive, activated T cells (but not LAG-3 negative T cells), and the close link of LAG-3 to several autoimmune diseases. The described approach is distinct from the use of the T cell modulator CTLA-4-Ig abatacept, which does not act to activate immune checkpoint pathways, but rather inhibits costimulatory pathways. Thus, the disclosure provides data supporting conversion of a normally antagonist LAG-3 antibody into an agonist that triggers LAG-3 inhibitory function through the described BiTS. The described BiTS approach can be combined with other autoimmune immunotherapies, such as those targeting B cell or cytokines (in a form of multi-specific antibody or combinational therapies).

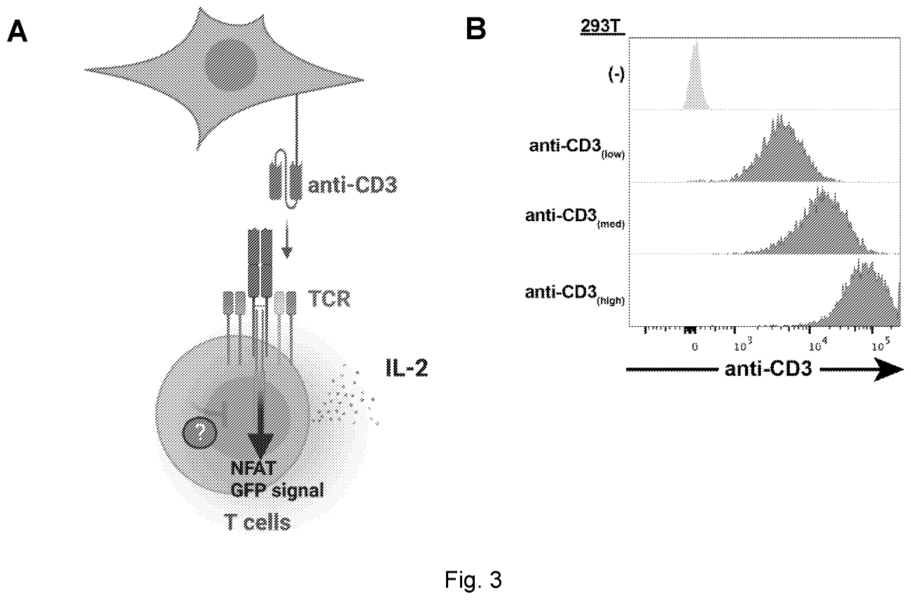

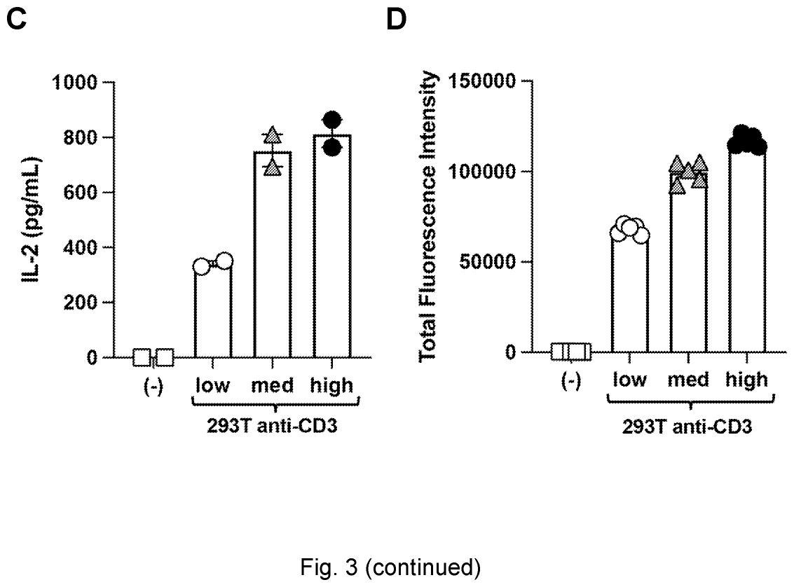

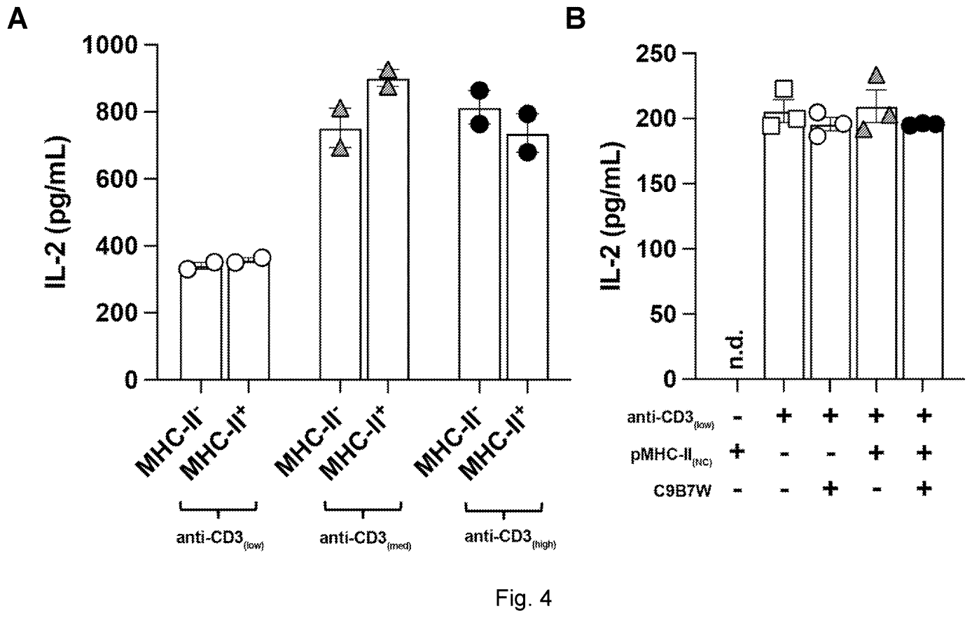

In an aspect, the disclosure provides a novel functional system involving an artificial antigen presenting cell (aAPC) and a mouse T cell hybridoma to differentiate the contribution of MHC-II on TCR and LAG-3. For this system this we expressed a membrane tethered anti-mouse CD3ε single chain variable fragment (scFv) that served as the TCR-CD3 activation signal and used MHC-II covalently bound to a non-cognate peptide not triggering TCR as the LAG-3 ligand (FIG. 2B). However, with this assay, LAG-3 mediated T cell inhibition by LAG-3 antibody blockade was not observed (FIG. 2B). We therefore also generated aAPC clones with low, medium, and high levels of membrane tethered anti-mouse CD3ε expression, which stimulated mouse T cell activation in different potencies as indicated by NFAT GFP reporter signal (FIG. 3A-B). With this system, we found over-expression of MHC-II fused with non-cognate peptide did not trigger LAG-3 mediated immune suppression on LAG-3 positive T cells under different strengths of TCR stimulation (FIG. 4A). And again, anti-LAG-3 antibody did not work in the absence or presence of MHC-II/non-cognate peptide (FIG. 4B). This unexpected result led to a determination that LAG-3 must be in proximity to the TCR-CD3 complex for its inhibitory function.

In the known system, MHC-II binds to both the TCR-CD3 complex and LAG-3 simultaneously, maintaining close contact between LAG-3 and the TCR-CD3 complex. To test the importance of TCR-CD3 proximity in LAG-3 function, we generated an in vitro APC/T cell co-culture system with or without enforced proximity between TCR-CD3 complex and LAG-3. We used a rapalog-inducible heterodimer system to bridge TCR-CD3 complex and LAG-3 by appending the FRB domain of mTOR to MHCII (covalently bound to non-cognate peptide), and FKBP12 to the membrane tethered anti-CD3ε (FIG. 5A). Incubation with a non-immunosuppressive rapalog, a small molecule, forms tight interaction with both FRB and FKBP12, and will hypothetically force proximity between MHC-II and the membrane tethered anti-CD3ε, and likewise LAG-3 and the TCR-CD3 complex. Experiments revealed that such heterodimers can trigger LAG-3 mediated inhibition (FIG. 5B), indicating that LAG-3 indeed works in proximity to the TCR.

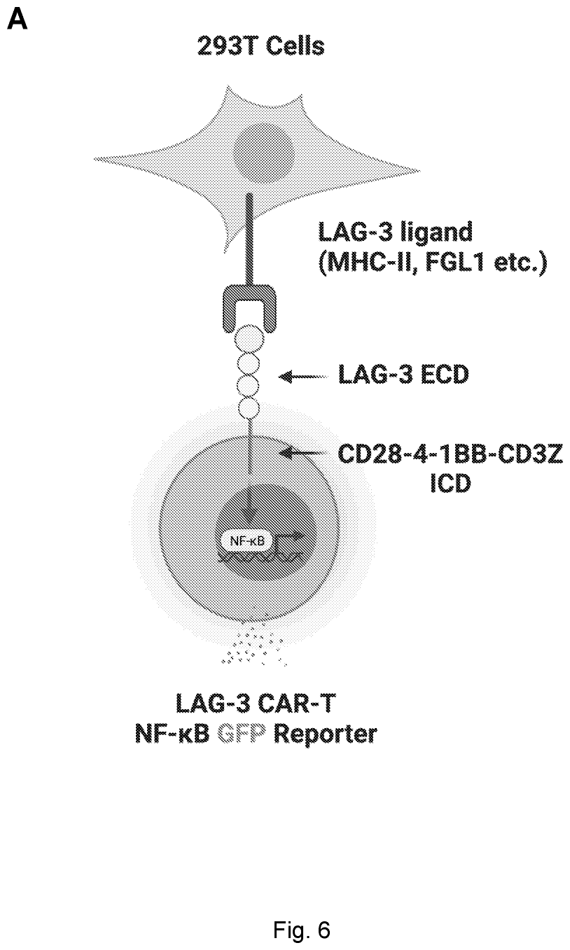

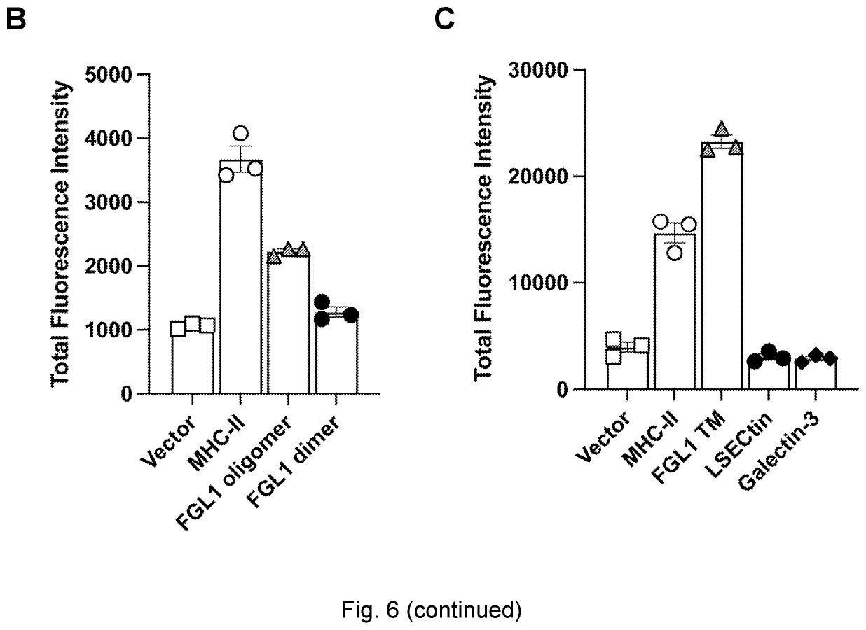

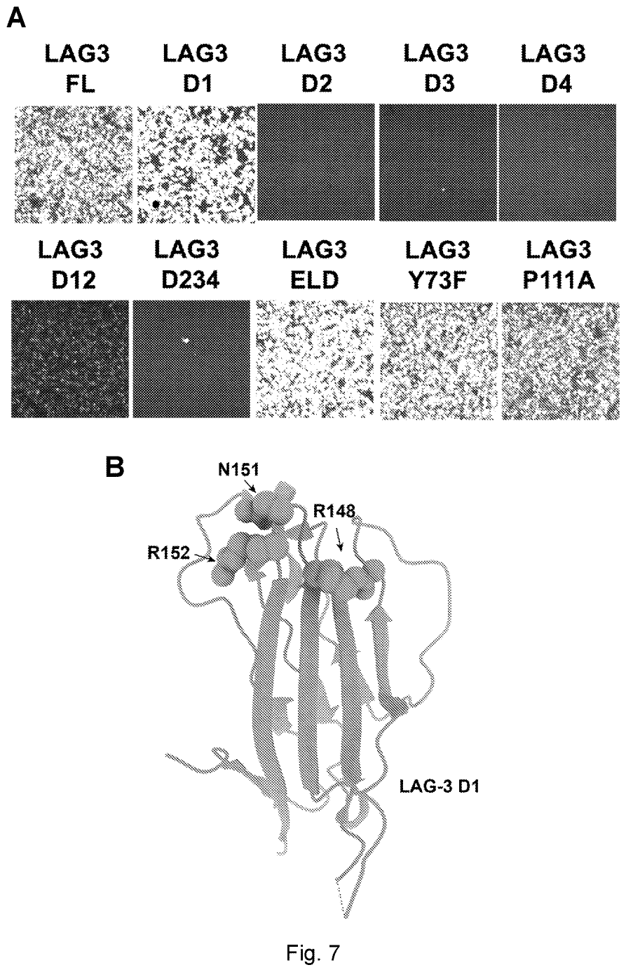

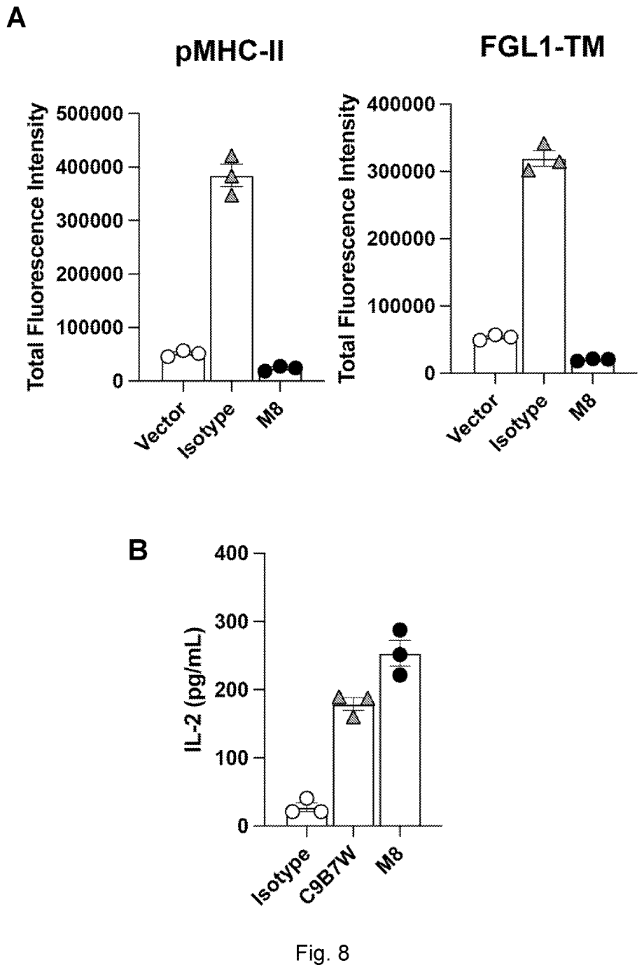

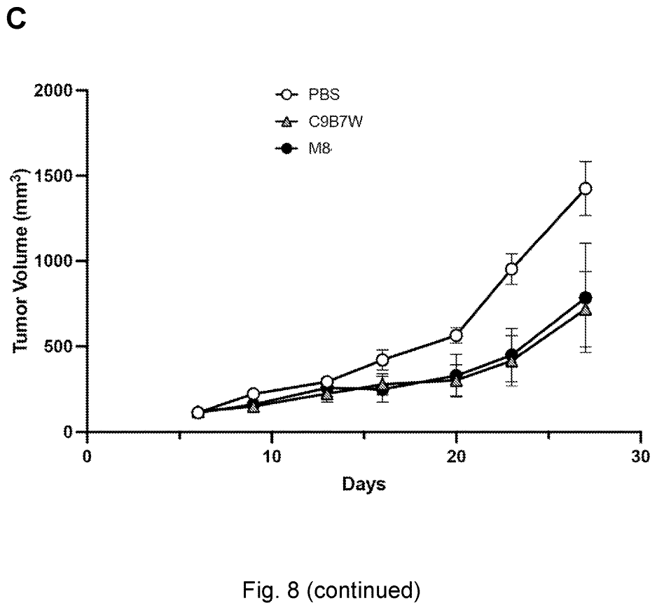

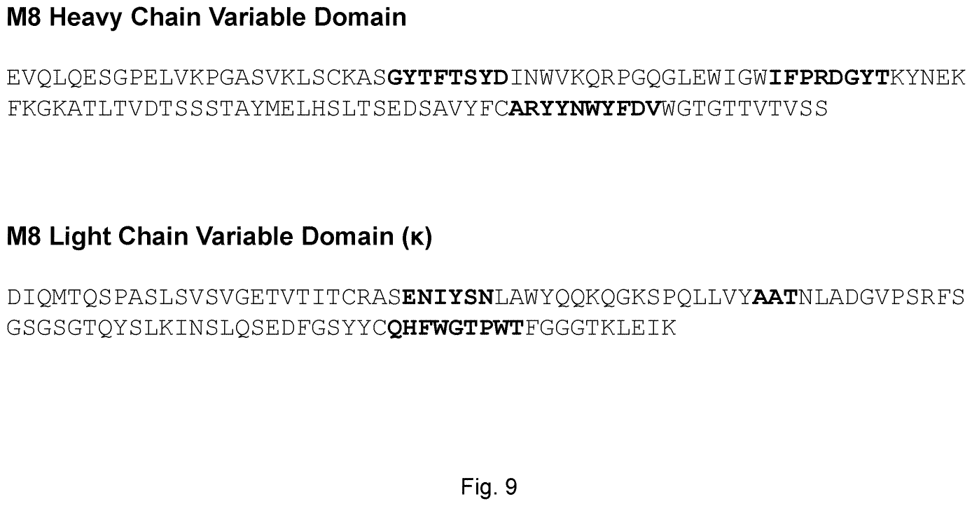

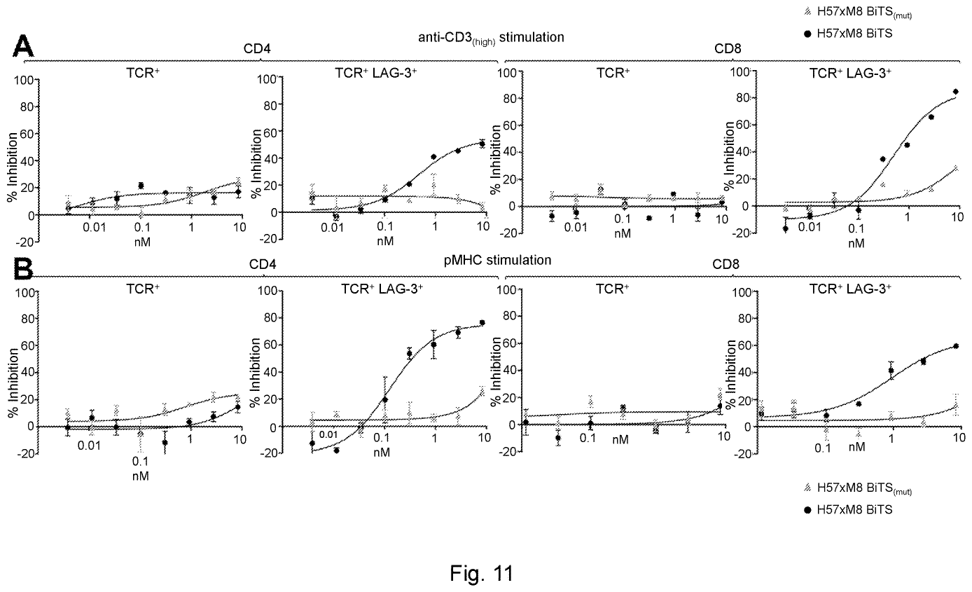

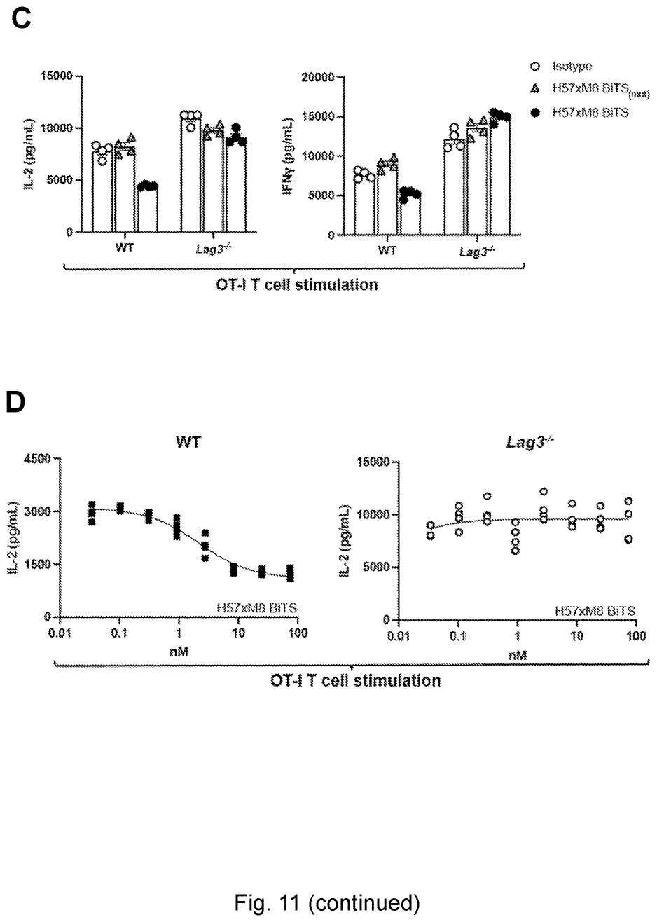

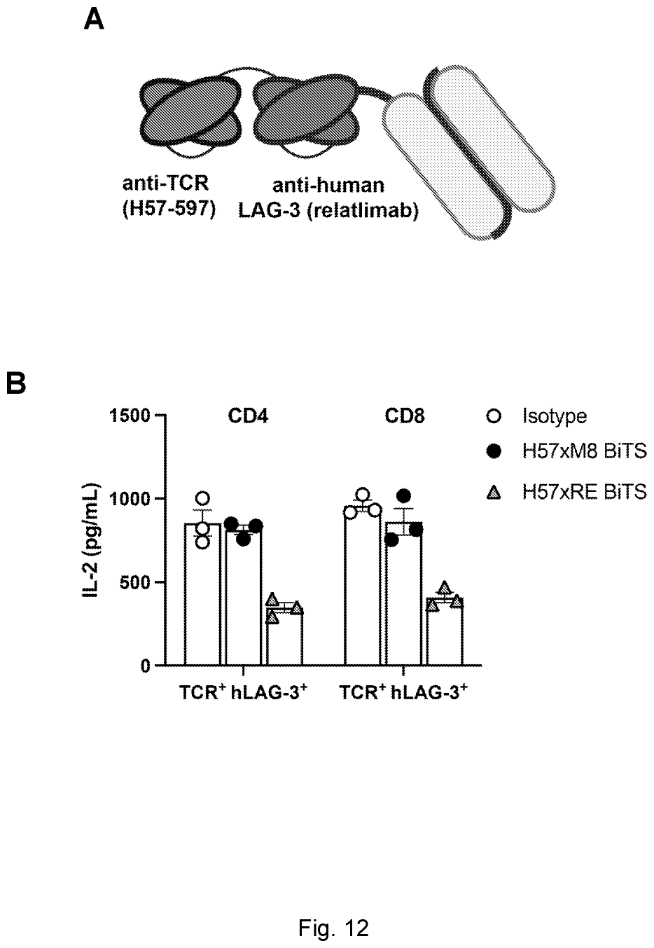

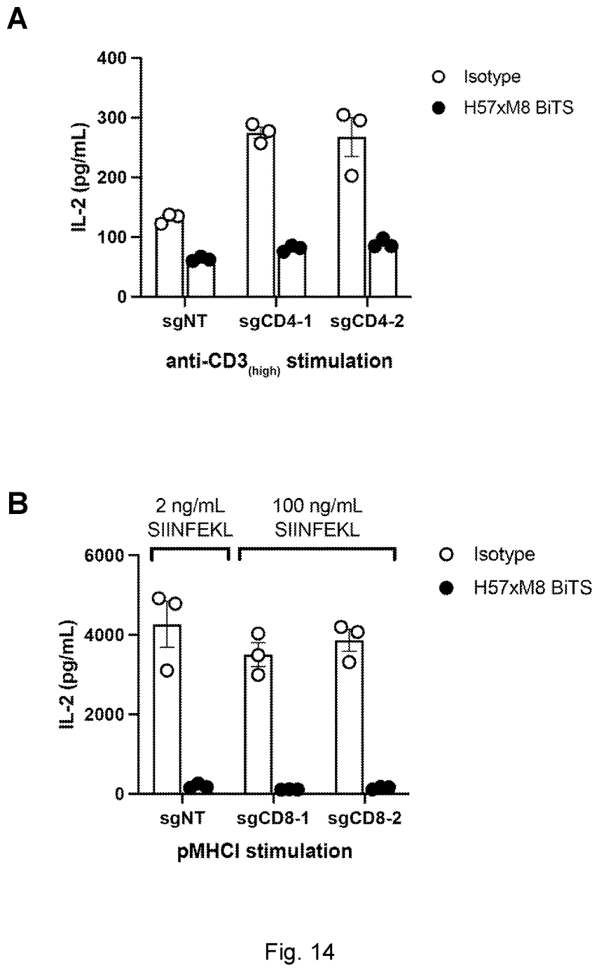

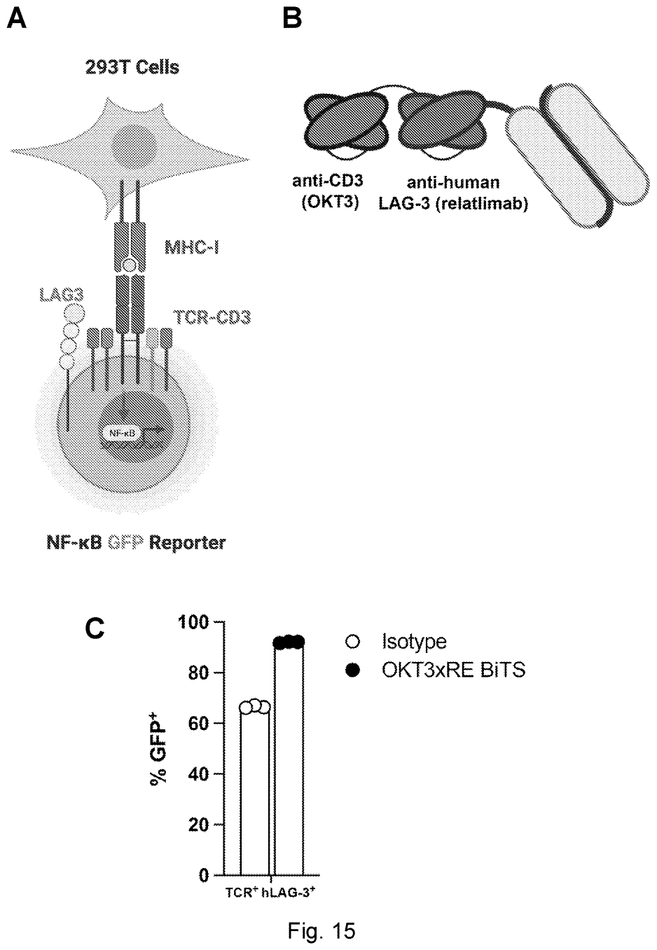

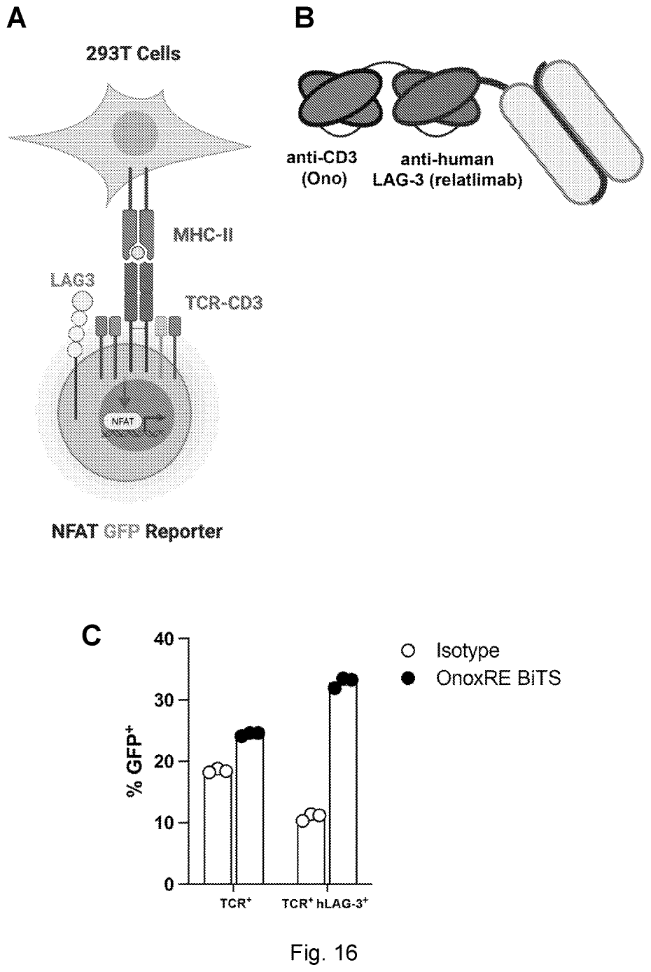

The disclosure includes a unique LAG-3 antibody screening system, a mouse LAG-3 Chimeric Antigen Receptor-like (CAR) NFκB-GFP reporter cell assay, to validate functional LAG-3 ligands and screen LAG-3 antibody with or without different ligand blocking capacity (FIG. 6A). This system can be used, for example, to identify other bispecific binding partners that could be used in the described methods. To develop the screening system, we combined the CD28/41BB/CD3ζ intracellular domains to the LAG-3 extracellular (ECD) and transmembrane (TM) domains and over-expressed the chimera on Jurkat NFκB-GFP reporter cells. Among all the known LAG-3 ligands, we found that only MHC-II and FGL1 (membrane-tethered or oligomeric form) are capable of triggering LAG-3-CAR activation (FIG. 6B-C). Using this LAG-3 CAR reporter system, we have identified several antibodies that can function as described bispecific antibodies. In non-limiting examples, data presented in this disclosure demonstrate that the hybridoma generated anti-mouse LAG-3 antibody (M8) is capable of blocking both MHC-II and FGL1 mediated signaling (FIG. 7A-B). This domain 1 antibody binds to epitopes outside of the typical loop region (68-91aa) of LAG-3 and showed potent function in reversing LAG-3 mediated immune suppression that is superior to C9B7W anti-mouse LAG-3 domain 2 antibody (FIG. 7-8). The disclosure therefore includes designing and using other BiTS that tether the LAG-3 receptor to a TCR complex on the same T cell. This prepares the TCR complex for inhibition upon antigen-induced TCR activation, which is expected to promote LAG-3 proximity to TCR (in cis) and mediate LAG-3⁺ positive T cell suppression. Based on this M8 anti-mouse LAG-3 antibody clone (FIG. 9), we developed an anti-mouse TCRβ (H57-597 clone, commercially available)/M8 bispecific single-chain fragment variable (ScFv) antibody (H57xM8 BiTS) that induces LAG-3 proximity to the TCR-CD3 complex (FIG. 10). We found H57xM8 BiTS can suppress both CD4 and CD8 T cell activation in an LAG-3 dependent manner (FIG. 11). Moreover, we also found anti-mouse TCRβ (H57-597 clone)/anti-human LAG-3 (Relatlimab from BMS) BiTS (H57xRE BiTS) could also potently suppress human LAG-3 positive CD4 or CD8 T cells (FIG. 12). The described results support the present approach for using anti-LAG-3 antagonist to enhance T cell activation by converting the LAG-3 antagonist into a LAG-3 agonist in a BiTS format by fusion with an anti-TCR antibody (FIGS. 10-12). We further demonstrate that the H57xM8 BiTS inhibits the TCR-CD3 complex and does not affect coreceptor signaling (FIG. 14). When using anti-human CD3 antibodies in the BiTS format, we have found that these BiTS activate rather than suppress T cell activity, suggesting the H57-597 epitope is important (FIG. 15-16). FIG. 15 uses a binding partner (that is not the LAG-3 binding partner) that binds to an epitope within the human CD3 complex. This binding partner is the OKT3 monoclonal antibody that is known in the art and is commercially available from, such as from BioXCell Catalog #BE0001-2. Likewise, FIG. 16 uses another anti-CD3 antibody that binds to an epitope distinct from OKT3. This binding partner is the anti-CD3 antibody from Ono Pharmaceutical Co., Ltd. described in US20220281977A1 from which the description of this antibody is incorporated herein by reference.

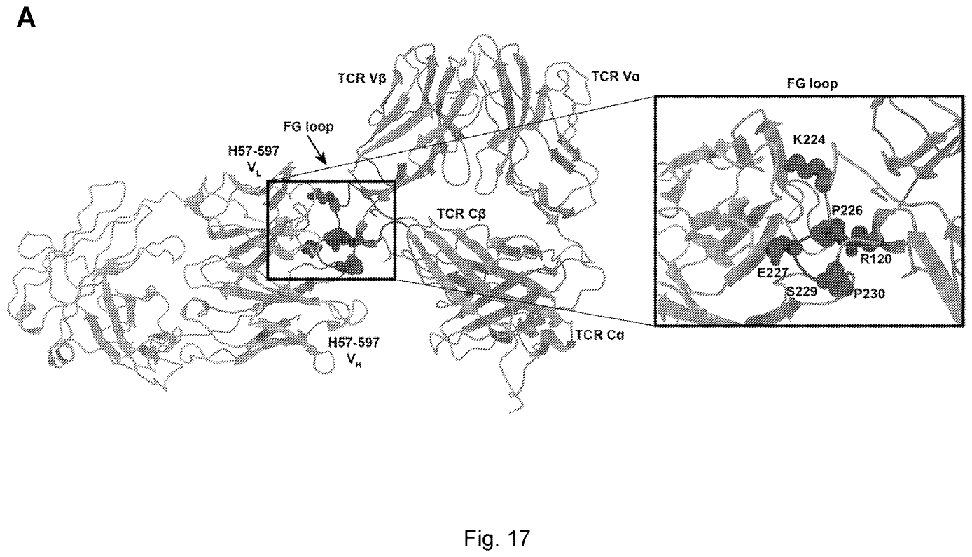

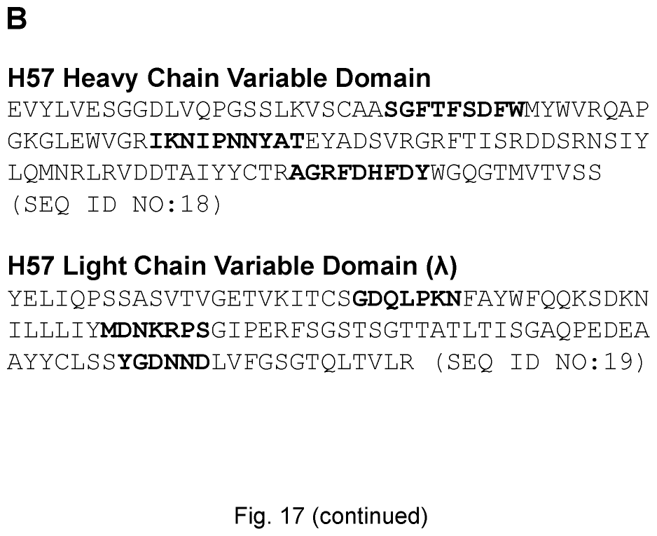

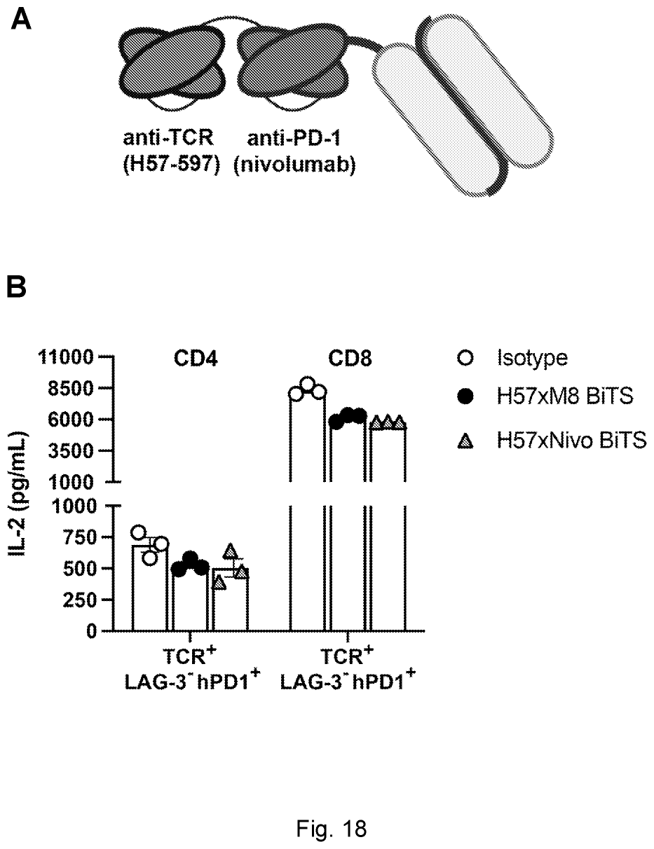

The H57-597 antibody targets the FG loop within the TCRβ constant chain (FIG. 17). Furthermore, we found that the anti-mouse TCRβ (H57-597 clone)/anti-human PD-1 (Nivolumab) cannot suppress T cells, suggesting that proximity induction via LAG-3 is important (FIG. 18). The disclosure includes BiTS that comprise anti-LAG-3 ScFv fused to other available TCR-CD3 ScFv antibodies beyond targeting TCR, for example, to CD3ε or CD6 that has been suggested to be closely associated with TCR-CD3 complex. In general, the disclosure includes use of anti-CD3ε antibodies with weak or no T cell activation.

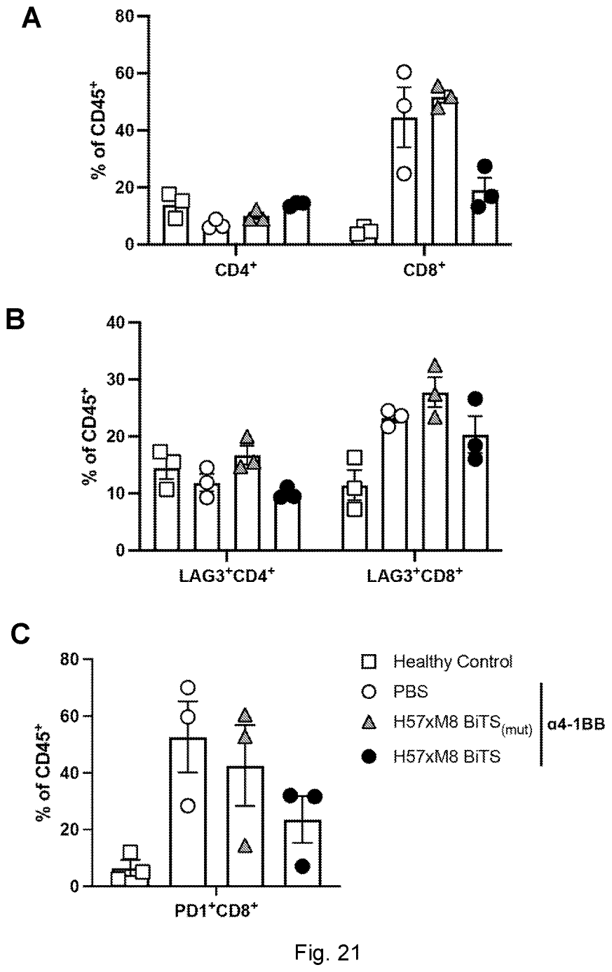

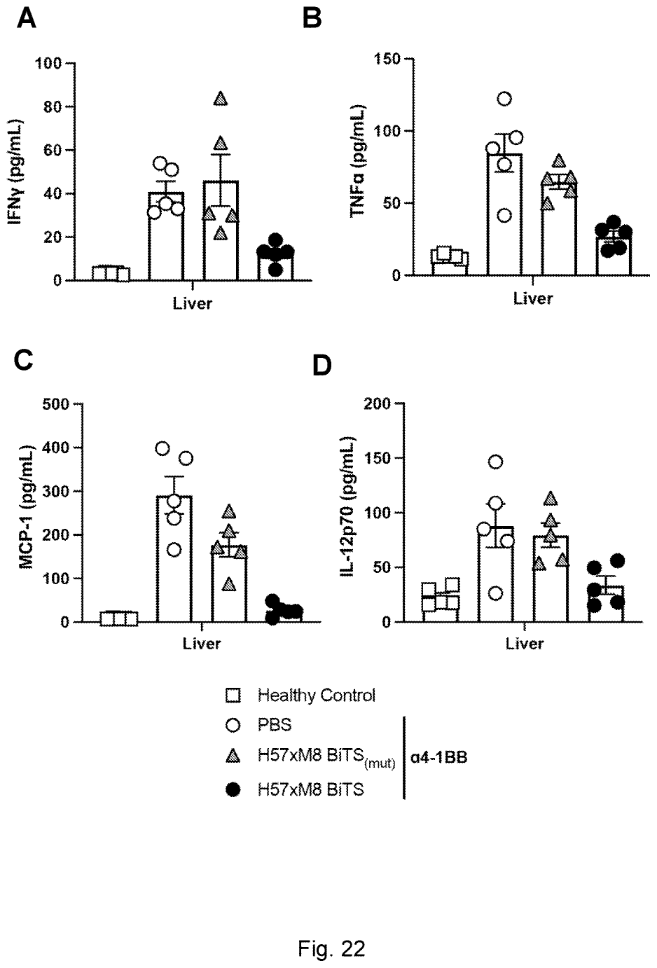

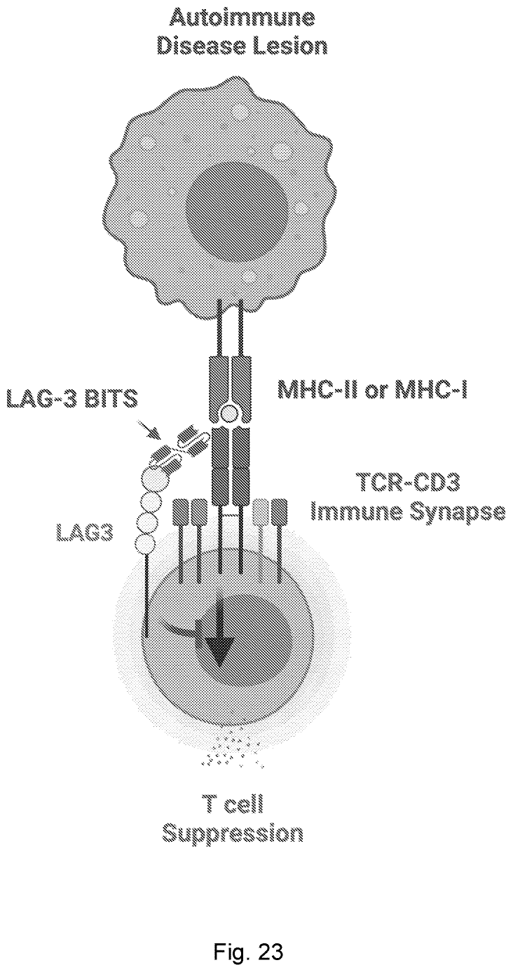

Given the potent function of the anti-LAG-3/TCR BiTS in the suppression of both CD4 and CD8 T cell activation in an LAG-3 dependent manner, the disclosure includes use of the described bispecific binding partners for treating a wide array of autoimmune disorders, as further described below. In a non-limiting demonstration, we tested H57xM8 BiTS in a MOG peptide-induced, CD4-driven experimental autoimmune encephalitis, a mouse model of multiple sclerosis. The described BiTS greatly ameliorated EAE disease symptoms, even at a low dose (1 mg/kg) for 5 daily doses (FIG. 13). We tested the H57xM8 BiTS in an autoimmune diabetes model and found that the described BiTS protected against the development of diabetes (FIG. 18). In an autoimmune hepatitis model induced by anti-41BB, we found that the H57xM8 BiTS protected against the development of hepatitis (FIG. 20). Moreover, the described BiTS did not deplete LAG-3⁺ T cells in this model (FIG. 21). When compared to the mutant version of the H57xM8 BiTS, the H57xM8 BiTS was more potent in suppressing inflammatory cytokine production within the liver (FIG. 22). These data demonstrate feasibility of use of the BiTS approach in T cell involved autoimmune diseases. LAG-3 has been closely associated in the pathogenesis of EAE, diabetes and arthritis, and soluble LAG-3 ligand FGL1 has demonstrated therapeutic effects in mouse arthritis. It is expected that the described BiTS may also synergize with existing treatment options, such as with anti-BAFF blockade (for lupus), and with CTLA-4-Ig (for arthritis).

While the bispecific binding partners of this disclosure are as described as such, the binding partners may be further modified to be, for example, tri-specific, and thus may specifically bind to another target, in addition to the TCR component and the LAG-3 component. Accordingly, a bispecific binding partner includes a binding partner that binds to at least to two different described targets. Thus, components of bispecific binding partners of this disclosure can be provided as intact immunoglobulins or as fragments of immunoglobulins, including but not necessarily limited to antigen-binding (Fab) fragments, Fab′ fragments, (Fab′)₂ fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), isolated complementary determining regions (CDRs), single-chain variable fragment (scFv), and other antibody fragments that retain antigen binding function. In embodiments, a chimeric antigen receptor (CAR) of this disclosure comprises scFv that comprises heavy and light chain variable regions. As is known in the art for previously described CARs, the scFv is present in a contiguous polypeptide that further comprises a CD3ζ chain and a costimulatory domain. In embodiments, the costimulatory domain comprises a 4-1BB costimulatory domain or a CD28 costimulatory domain. A CAR may also contain a co-receptor hinge sequence, such as a CD8 a co-receptor hinge sequence.

In embodiments, binding partners of this disclosure may comprise a constant region, e.g., an Fc region. Any isotype of constant region can be included. However, the disclosure includes the proviso that the presently described approach does not require Fc/Fcr interactions.

In certain examples, the described bispecific binding partner may comprise linking amino acids that connect the first binding component that binds with specificity to a TCR component or other described protein that is in proximity with the TCR, and the second binding component that binds with specificity to LAG-3. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, or 20 amino acids.

Binding partners and pharmaceutical compositions comprising the binding partners can be administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. The compositions may be administered parenterally or enterically. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated.

The disclosure includes binding partners for use in diagnostic and prophylactic approaches. For therapeutic approaches, in certain embodiments, binding partners may be delivered as mRNA or DNA polynucleotides that encode the binding partners. It is considered that administering a DNA or RNA encoding any binding partner described herein is also a method of delivering such binding partners to an individual or one or more cells. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus.

In embodiments, the disclosure includes modified cells that are modified such that they express a described binding partner. In embodiments, the modified cells modified lymphocytes. In embodiments, the modified cells are T cells, natural killer cells, or macrophages. In embodiments, the modified cells are modified stem cells. In embodiments, the modified cells are totipotent, pluripotent, or multipotent stem cells. In embodiments, the described cells are used as therapeutics agents.

In embodiments, the individual in need of a composition of this disclosure has been diagnosed with or is suspected of having an autoimmune disease. In embodiments, the autoimmune disease is any of systemic lupus erythematosus, rheumatoid arthritis, chronic inflammation, celiac disease, Crohn's disease, colitis, diabetes mellitus type 1, inflammatory bowel disease, autoimmune encephalitis, eosinophilic fasciitis, eosinophilic gastroenteritis, eosinophilic esophagitis, multiple sclerosis (MS), including but not limited to Relapsing-Remitting MS, Secondary-Progressive MS, Primary-Progressive MS, and Progressive-Relapsing MS, or gastritis, Graves' disease, hypogammaglobulinemia, idiopathic inflammatory demyelinating diseases, thrombocytopeniaurpura, myasthenia gravis, pernicious anemia, psoriasis, Sjögren's syndrome, ulcerative colitis, graft versus host disease (GVHD) or any autoimmune disease that is characterized by type II, III or IV hypersensitivity, Polymyalgia rheumatic, Addison's disease, Behcet's disease, scleroderma (systemic sclerosis), Autoimmune pancreatitis, Autoimmune hemolytic anemia, Hypoparathyroidism, Guillain-Barre syndrome, Reactive arthritis, or Sarcoidosis. In embodiments, the individual has been diagnosed with or is suspected of one or a combination of primary-progressive multiple sclerosis (PPMS), relapsing-remitting MS (RRMS), secondary-progressive MS (SPMS), or progressive-relapsing MS (PRMS).

In embodiments, an effective amount of one or more binding partners is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight, and gender of the patient, desired duration of treatment, method of administration, time and frequency of administration, drug combination(s), reaction sensitivities, and/or tolerance/response to therapy.

In embodiments, the binding partners are produced by host cells by way of recombinant expression vectors and cell cultures. In embodiments, the cell cultures include prokaryotic cells or eukaryotic cells. In embodiments, the cell cultures are mammalian cells. In embodiments, the cells are CHO cells. In embodiments, the cells are HEK293 cells or their derivatives.

Kits comprising the binding partners, and/or cell cultures expressing the binding partners, are provided by this disclosure. In general, the kits comprise one or more sealed containers that contain the binding partners, or cells expressing them. Instructions for using the binding partners for therapeutic and/or prophylactic purposes can be included in the kits.

Other embodiments of the disclosure will be apparent to those skilled in the art from consideration of the specification and practice of the disclosure disclosed herein. It is intended that the specification and examples be considered as exemplary only.

1. A binding partner having a first binding component that specifically binds to a T cell receptor (TCR) component or to another protein that is proximal to a TCR chain, wherein the TCR component is optionally a TCR beta (TCRβ) chain, and a second binding component that binds with specificity to LAG-3.

2. The binding partner of claim 1, wherein the first and second binding components are in the form of a bispecific antibody-based T cell silencer (BiTS).

3. The binding partner of claim 2, wherein the first and second components are in the form of the BiTS, and wherein binding of the BiTS to a T cell suppresses activity of the T cell

4. The binding partner of claim 1, wherein the TCR component is the TCRβ chain.

5. A method comprising administering to an individual who has an autoimmune disorder a binding partner having a first binding component that specifically binds to a T cell receptor (TCR) chain or to another protein that is proximal to a TCR chain, and a second binding component that binds with specificity to LAG-3.

6. The method of claim 5, wherein the first and second binding components are in the form of a bispecific antibody-based T cell silencer (BiTS).

7. The method of claim 6, wherein the first binding component binds to a T cell receptor (TCR) chain.

8. The method of claim 7, wherein the TCR chain is a TCRβ chain.

9. The method of claim 5, wherein the autoimmune disease is any of: systemic lupus erythematosus, rheumatoid arthritis, chronic inflammation, celiac disease, Crohn's disease, colitis, diabetes mellitus type 1, inflammatory bowel disease, autoimmune encephalitis, eosinophilic fasciitis, eosinophilic gastroenteritis, eosinophilic esophagitis, multiple sclerosis (MS), including but not limited to Relapsing-Remitting MS, Secondary-Progressive MS, Primary-Progressive MS, and Progressive-Relapsing MS, or gastritis, Graves' disease, hypogammaglobulinemia, idiopathic inflammatory demyelinating diseases, thrombocytopenia purpura, myasthenia gravis, pernicious anemia, psoriasis, Sjögren's syndrome, ulcerative colitis, graft versus host disease (GVHD) or any autoimmune disease that is characterized by type II, III or IV hypersensitivity, Polymyalgia rheumatic, Addison's disease, Behcet's disease, scleroderma (systemic sclerosis), Autoimmune pancreatitis, Autoimmune hemolytic anemia, Hypoparathyroidism, Guillain-Barré syndrome, Reactive arthritis, or Sarcoidosis.

10. The method of claim 9, wherein progression of the autoimmune disease is inhibited.

11. The method of claim 9, wherein severity of or one more symptoms of the autoimmune disease is reduced.

12. A pharmaceutical composition comprising a binding partner of claim 1.

13. The pharmaceutical composition of claim 12, wherein the first and second binding components are in the form of a bispecific antibody-based T cell silencer (BiTS).

14. The pharmaceutical composition of claim 13, wherein the TCR component is the TCRβ chain or a TCR-CD3 complex.

15. A modified cell that is modified to express the binding partner of claim 1.

16. The modified cell of claim 15, wherein the binding partner is in the form of a bispecific antibody-based T cell silencer (BiTS).

17. The modified cell of claim 16, wherein the binding partner is in the form of a bispecific antibody-based T cell silencer (BiTS).

18. The modified cell of claim 17, wherein the TCR component is a TCR beta (TCRβ) chain

19. A polynucleotide encoding the binding partner of claim 1.

20. The polynucleotide of claim 19, wherein the binding partner is in the form of a bispecific antibody-based T cell silencer (BiTS), and wherein the polynucleotide is comprised by an expression vector.
